(19) United States
(12) Patent Application Publication
(10) Pub. No.: US 2002/0055499 A1
(43) Pub. Date: May 9, 2002

(54) Integrin receptor antagonists

(76) Inventors: Dirk Heerding, Malvern, PA (US); James Martin Samanen, Phoenixville, PA (US)

Correspondence Address: GLAXOSMITHKLINE, Corporate Intellectual Property - UW 2220, P.O. Box 1539, King of Prussia, PA, 19406-0939, US

(21) Appl. No.: 10/020,012

(22) Filed: Dec. 12, 2001

Related U.S. Application Data
(63) Continuation of application No. 09/485,178, filed on Feb. 3, 2000.
Application No. 09/485,178 is a 371 of international application No. PCT/US98/16246, filed on Aug. 4, 1998.
(60) Provisional application No. 60/063,526, filed on Oct. 29, 1997.
(60) Provisional application No. 60/054,703, filed on Aug. 4, 1997.

(51) Int. Cl. A61K 31/553; A61K 31/554; A61K 31/55; A61K 31/4745; A61K 31/407

(52) U.S. Cl. 514/211.05; 514/217; 514/212.07; 514/291; 514/411; 514/431; 514/450; 540/488; 540/490; 540/547; 540/523; 540/586; 546/80; 548/430; 549/12; 549/349

(57) ABSTRACT

Compounds of the formula (I) are disclosed which are dual fibrinogen receptor and vitronectin receptor antagonists and are useful in the treatment of atherosclerosis, in the prevention of restenosis and in the prevention of tumor metastasis and tumor growth:

[chemical structure]

### FIELD OF THE INVENTION

This invention relates to pharmaceutically active compounds which bind to integrins, such as dual antagonists of the vitronectin receptor and the fibrinogen receptor. Such compounds are useful for inhibiting platelet aggregation, treating restenosis and atherosclerosis and are useful in vascular remodeling.

### BACKGROUND OF THE INVENTION

Integrins are a family of heterodimeric proteins which generally mediate cell adhesion. Typical of such proteins are the vitronectin receptor (an αᵥβ₃ heterodimer) and the fibrinogen receptor (an (α_{IIb}β₃ heterodimer). The natural ligands of these receptors (e.g., vitronectin and fibrinogen) have been found to share a common -Arg-Gly-Asp- amino acid sequence, which appears to be critical for binding. In fact, many of the integrin receptors appear to cross react with ligands which possess such an amino acid sequence. For instance, the α_{IIb}β₃ receptor reacts with fibronectin and vitronectin, thrombospondin and von Willebrand factor, as well as fibrinogen. Functionally fibrinogen, a dimer having two binding sites for α_{IIb}β₃, reacts with activated receptors found on the surface of platelets. The binding of α_{IIb}β₃ receptors on adjacent platelets, by fibrinogen leads to crosslinking and is considered to be a major factor in platelet aggregation. Compounds which inhibit the binding of the α_{IIb}β₃ receptor to fibrinogen have been shown to inhibit the platelet aggregation in vitro, and thrombus formation in vivo. See, for instance, EP-A 0 341 915. Animal models have demonstrated that inhibition of the α_{IIb}β₃ receptor may reduce neointimal hyperplasia after angioplasty. Friedman, et al., J. Clin. Invest., 1977, 60, 1191. Additionally, studies have shown that α_{IIb}β₃ antagonists have been able to prevent tumor invasion and metastasis in vitro. Honn, et al., Cancer Res., 1997, 57, 2522.

The vitronectin receptor αᵥβ₃ is expressed on a number of cells, including endothelial, smooth muscle, osteoclast, and tumor cells, and, thus, it has a variety of functions. It has been reported that the αᵥβ₃ receptor expressed on human aortic smooth muscle cells stimulates their migration into neointima, which leads to the formation of atherosclerosis and restenosis after angioplasty. Brown, et al., Cardiovascular Res., 1994, 28, 1815. Additionally, a study has shown that a αᵥβ₃ antagonist is able to promote tumor regression by inducing apoptosis of angiogenic blood vessels. Brooks, et al.. Cell, 1994, 79, 1157. Thus, agents that block the vitronectin receptor and the fibrinogen receptor would be useful in treating diseases mediated by these receptors, such as atherosclerosis, restenosis (Topoi. er al., J. Am. Coll. Cardiol., 1996, 28, 1643.) and cancer.

It has now been discovered that certain new compounds are useful as integrin receptor antagonists, in particular the compounds of the present invention are useful as dual α_{IIb}β₃/αᵥβ₃ receptor antagonists.

### SUMMARY OF THE INVENTION

It is an object of this invention to provide compounds of the formula (I), as described hereinafter, which have pharmacological activity for the inhibition of integrin receptors. It is an object of this invention to provide a template which may be suitably substituted to provide selective binding for specific integrin receptors, especially the fibrinogen (α_{IIb}β₃) and the vitronectin (αᵥβ₃) receptors relative to each other and other integrin receptors.

This invention is also a pharmaceutical composition comprising a compound according to formula (I) and a pharmaceutically carrier.

This invention is also a method of treating diseases in which the pathology may be modified by binding to integrin receptors, especially the vitronectin and the fibrinogen receptors. In a particular aspect, the compounds of this invention are useful for treating atherosclerosis and restenosis, and conditions in which it is desirable to inhibit platelet aggregation, such as stroke, transient ischemia attacks, myocardial infarction and rethrombosis following thrombolytic therapy.

### DETAILED DESCRIPTION

This invention comprises compounds of formulae (I) or (II):

[chemical structure]

wherein:

A is NR′, CHR′, O or S;

A₁ is C or N;

X¹ and X² are C═O or CHR′, with the proviso that only one of X¹ or X² is C═O;

X³ is CHR′, NR′, O or S;

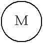

is a five- or six-membered heteroaromatic or six-membered aromatic ring optionally substituted by R¹¹;

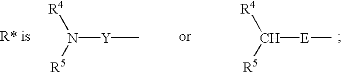

E is O, S, C(O) or C(S);

Y is C(O), C(S) or CH₂;

R¹ is R⁷, or D-C₀₋₄alkyl, D-C₂₋₄alkenyl, D-C₂₋₄alkynyl, D-C₃₋₄oxoalkenyl, D-C₃₋₄oxoalkynyl, D-C₁₋₄aminoalkyl, D-C₃₋₄aminoalkenyl, D-C₃₋₄aminoalkynyl, optionally substituted by any accessible combination of one or more of R¹¹ or R⁷;

R² is —(CH₂)ₜ—CO₂R³;

R³ is H, C₁₋₆alkyl or (CHR′)ₙ—Ar;

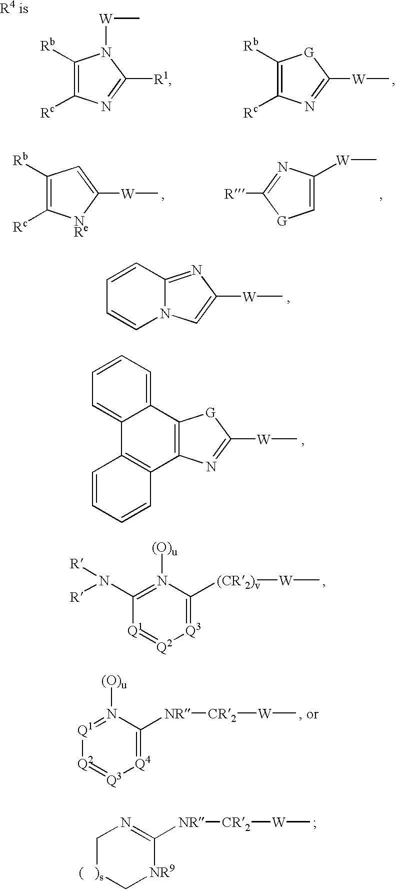

W is —(CHR^{g})ₐ—U— (CHR^{g})_{b}—;

U is absent or CO, CR²₂, C(═CR^{g}₂), S(O)ₖ, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂N═N, NR^{g}NR^{g}, NR^{g}CR^{g}₂, NR^{g}CR^{g}₂, CR^{g}₂O, OCR^{g}₂, C≡C or CR^{g}═CR^{g};

G is NR^{e}, S or O;

R^{g} is H, C₁₋₆alkyl, Het-C₀₋₆alkyl, C₃₋₇cycloalkyl-C₀₋₆alkyl or Ar—C₀₋₆alkyl;

R^{k} is R^{g}, —C(O)R^{g}, or —C(O)OR^{f};

R¹ is is H, C₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂,

R^{f} is H, C₁₋₆alkyl or Ar—C₁₋₆alkyl;

R^{e} is H, C₁₋₆alkyl, Ar—C₁₋₆alkyl, Het-C₁₋₆alkyl, C₃₋₇cycloalkyl-C₁₋₆alkyl, or (CH₂)ₖCO₂R^{g}.

R^{b} and R^{c} are independently selected from H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂, or R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy;

Q¹, Q², Q³ and Q⁴ are independently N or C—R^{y}, provided that no more than one of Q¹, Q², Q³ and Q⁴ is N;

R^{y} is H, halo, —OR^{g}, —SR^{g}, —CN, —NR^{g}R^{k}, —NO₂, —CF₃, CF₃S(O)ᵣ-, —CO₂R^{g}, —COR^{g} or —CONR^{g}₂, or C₁₋₆alkyl optionally substituted by halo, —OR^{g}, —SR^{g}, —CN, —NR^{g}R″, —NO₂, —CF₃, R′S(O)ᵣ-, —CO₂R^{g}, —COR^{g} or —CONR^{g}₂;

R⁵ is W′-(CR′₂)_{q}-Z-(CR′R¹⁰)ᵣ-U′-(CR′)ₛ;

U′ is absent or CO, CR′₂, C(═CR′2), S(O)ₙ, O, NR′, CR′OR′, CR′(OR″)CR′₂, CR′₂CR′(OR″), C(O)CR′₂, CR′₂C(O), CONR′, NR′CO, OC(O), C(O)O, C(S)O, OC(S), C(S)NR′, NR′C(S), S(O)ₙNR′, NR′S(O)ₙ, N═N, NR′NR′, NR′CR′₂, NR′CR′₂, CR′₂O, OCR′₂, c≡c or CR′═CR′;

W′ is R′R″N—, R′R″NR′N—, R′R″NR′NCO—, R′₂NR′NC(═NR′)—,

[chemical structure]

X is N═CR′, C(O) or O;

Y is absent, S or O;

Z is (CH₂)ₜ, Het, Ar or C₃₋₇cycloalkyl;

R⁷ is —COR⁸, —COCR′₂R⁹, —C(S)R⁸, —S(O)ₘOR′, —S(O)ₘNR′R″, —PO(OR′), —PO(OR′)₂, —B(OR′)₂, —NO₂ and Tet;

R⁸ is —OR′, —NR′R″, —NR′SO₂R′, —NR′OR′, —OCR′₂C(O)OR′, —OCR′₂OC(O)—R′, —OCR′₂C(O)NR′₂ or CF₃;

R⁹ is —OR′, —CN, —S(O)ᵣR′, S(O)ₘNR′₂, —C(O)R′C(O)NR′₂ or —CO₂R′;

R¹⁰ is H, C₁₋₄alkyl or —NR′R″;

each R¹¹ independently is H, halo, —OR¹², —CN, —NR′R¹², —NO₂, —CF₃, CF₃S(O)ᵣ-, —CO₂R′, —CONR′₂, D-C₀₋₆alkyl-, D-C₁₋₆oxoalkyl-, D-C₂₋₆alkenyl-, D-C₂₋₆alkynyl-, D-C₀₋₆alkyloxy-, D-C₀₋₆alkylamino- or D-C₀₋₆alkyl-S(O)ᵣ-;

R¹² is R′, —C(O)R′, —C(O)NR′₂, —(O)OR¹⁵, —S(O)ₘR′ or S(O)ₘNR′₂;

R¹³ is R′, —CF₃, —SR′, or —OR′;

R¹⁴ is R′, C(O)R′, CN, NO₂, SO₂R′ or C(O)OR¹⁵;

R¹⁵ is H, C₁₋₆alkyl or Ar—C₀₋₄alkyl;

D is H, C₃₋₆cycloalkyl. Het or Ar;

R′ is H, C₁₋₆alkyl, Ar—C₀₋₆alkyl or C₃₋₆cycloalkyl-C₀₋₆alkyl;

R″ is R′, —C(O)R′ or —C(O)OR′;

R′″ is H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂;

n is 0 to 3;

q is 0 to 3;

t is 0 to 2;

a is 0, 1 or 2;

b is 0, 1 or 2;

k is 0, 1 or 2;

m is 1 or 2;

r is 0, 1 or 2;

s is 0, 1 or 2;

u is 0 or 1; and

v is 0 or 1;

or a pharmaceutically acceptable salt thereof.

Preferably, this invention comprises formula (I) compounds of formula (Ia):

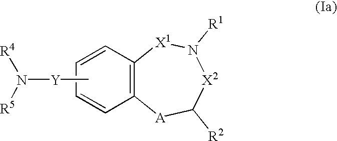

wherein:

A is NR′, CHR′, O or S;

X¹ and X² are C═O or CHR′, with the proviso that only one of X¹ or X² is C═O;

Y is C(O), C(S) or CH₂;

R¹ is R⁷, or D-C₀₋₄alkyl, D-C₂₋₄alkenyl, D-C₂₋₄alkynyl, D-C₃₋₄oxoalkenyl, D-C₃₋₄oxoalkynyl, D-C₁₋₄aminoalkyl, D-C₃₋₄aminoalkenyl, D-C₃₋₄aminoalkynyl, optionally substituted by any accessible combination of one or more of R¹¹ or R⁷;

R² is —(CH₂)ₜ—CO₂R³;

R³ is H, C₁₋₆alkyyl or (CHR′)ₙ—Ar;

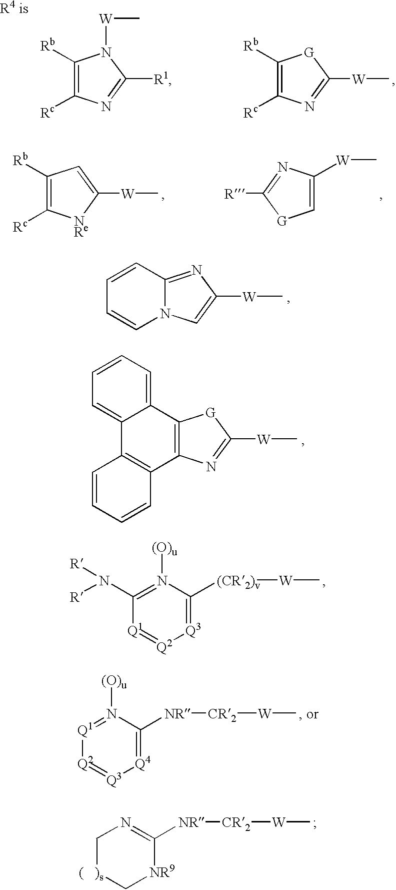

W is —(CHR^{g})ₐ—U— (CHR^{g})_{b}—;

U is absent or CO, CR^{g}₂, C(═CR^{g}₂), S(O)ₖ, O, NR_{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱ CO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, NR^{g}CR^{g}₂, CR^{g}₂O, OCR^{g}₂, C≡C or CR^{g}═CR^{g};

G is NR^{e}, S or O;

R^{g} is H, C₁₋₆alkyl, Het-C₀₋₆alkyl, C₃₋₇cycloalkyl-C₀₋₆alkyl or Ar—C₀₋₆alkyl;

R^{k} is R^{g}, —C(O)R^{g}, or —C(O)OR^{f};

Rⁱ is is H, C₁₋₆alkyl, Het-C₀₋₆alkyl, C₃₋₇cycloalkyl-C₀₋₆alkyl, Ar-C₀₋₆alkyl, or C₁₋₆alkyl substituted by one to three groups chosen from halogen, CN, NR^{g}₂, OR^{g}, SR^{g}, CO₂R^{g}, and CON(R^{g})₂;

R^{f} is H, C₁₋₆alkyl or Ar—C₁₋₆alkyl;

R^{e} is H, C₁₋₆alkyl, Ar—C₁₋₆alkyl, Het-C₁₋₆alkyl, C₃₋₇cycloalkyl-C₁₋₆alkyl, or (CH₂)ₖCO₂R^{g};

R^{b} and R^{c} are independently selected from H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂, or R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy;

Q¹, Q², Q³ and Q⁴ are independently N or C—R^{y}, provided that no more than one of Q¹, Q², Q³ and Q⁴ is N;

R^{y} is H, halo, —OR^{g}, —SR^{g}, —CN, —NR^{g}R^{k}, —NO₂, —CF₃, CF₃S(O)ᵣ-, —CO₂R^{g}, —COR^{g} or —CONR^{g}₂, or C₁₋₆alkyl optionally substituted by halo, —OR^{g}, —SR^{g}, —CN, —NR^{g}R′, —NO₂, —CF₃, R′S(O)ᵣ′, —CO₂R^{g}, —COR^{g} or —CONR^{g}₂;

R⁵ is W′-(CR′₂)_{q}-Z-(CR′R¹⁰)ᵣ-U′-(CR′₂)ₛ;

U′ is absent or CO, CR′₂, C(═CR′2), S(O)ₙ, O, NR′, CR′OR′, CR′(OR″)CR′₂, CR′₂CR′(OR″), C(O)CR′₂, CR′₂C(O), CONR′, NR′CO, OC(O), C(O)O, C(S)O, OC(S), C(S)NR′, NR′C(S), S(O)ₙNR′, NR′S(O)ₙ, N═N, NR′NR′, NR′CR′₂, NR′CR′₂, CR′₂O, OCR′₂, c≡c or CR′═CR′;

W′ is R′R″N—, R′R″NR′N—, R′R″NR′NCO—, R′₂NR′NC(═NR′)—,

[chemical structure]

X is N═CR′, C(O) or O;

Y is absent, S or O;

Z is (CH₂)ₜ, Het, Ar or C₃₋₇cycloalkyl;

R⁷ is —COR⁸, —COCR′₂R⁹, —C(S)R⁸, —S(O)ₘOR′, —S(O)ₘNR′R″, —PO(OR′), —PO(OR′)₂, —B(OR′)₂, —NO₂ and Tet;

R⁸ is —OR′, —NR′R″, —NR′SO₂R′, —NR′OR′, —OCR′₂C(O)OR′, —OCR′₂OC(O)—R′, —OCR′₂C(O)NR′₂ or CF₃;

R⁹ is —OR′, —CN, —S(O)ᵣR′, S(O)ₘNR′₂, —C(O)R′ C(O)NR′₂ or —CO₂R′;

R¹⁰ is H, C₁₋₄alkyl or —NR′R″;

R¹¹ is H, halo, —OR¹², —CN, —NR′R¹², —NO₂, —CF₃, CF₃S(O)ᵣ-, —CO₂R′, —CONR′₂, D-C₀₋₆alkyl-, D-C₁₋₆oxoalkyl-, D-C₂₋₆alkenyl-, D-C₂₋₆alkynyl-, D-C₀₋₆alkyloxy-, D-C₀₋₆alkylamino- or D-C₀₋₆alkyl-S(O)ᵣ-;

R¹² is R′, —C(O)R′, —C(O)NR′₂, —C(O)OR¹⁵, —S(O)ₘR′ or S(O)ₘNR′₂;

R¹³ is R′, —CF₃, —SR′, or —OR′;

R¹⁴ is R′, C(O)R′, CN, NO₂, SO₂R′ or C(O)OR¹⁵;

R¹⁵ is H, C₁₋₆alkyl or Ar—C₀₋₄alkyl;

D is H, C₃₋₆cycloalkyl, Het or Ar;

R′ is H, C₁₋₆alkyl, Ar—C₀₋₆alkyl or C₃₋₆cycloalkyl-C₀₋₆alkyl;

R″ is R′, —C(O)R′ or —C(O)OR′;

R′″ is H, C₁₋₆alkyl, Ar—C₀₋₆alkyl. Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂;

n is 0 to 3;

q is 0 to 3;

t is 0 to 2;

a is 0, 1 or 2;

b is 0, 1 or 2;

k is 0, 1 or 2;

m is 1 or 2;

r is 0, 1 or 2;

s is 0, 1 or 2;

u is 0 or 1; and

v is 0 or 1;

or a pharmaceutically acceptable salt thereof.

Also included in this invention are pharmaceutically acceptable addition salts, complexes or prodrugs of the compounds of this invention. Prodrugs are considered to be any covalently bonded carriers which release the active parent drug according to formula (I) in vivo. In cases wherein the compounds of this invention may have one or more chiral centers, unless specified, this invention includes each unique nonracemic compound which may be synthesized and resolved by conventional techniques. In cases in which compounds have unsaturated carbon-carbon double bonds, both the cis (Z) and trans (E) isomers are within the scope of this invention. In cases wherein compounds may exist in tautomeric forms, such as keto-enol tautomers, such as

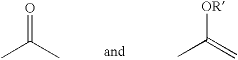

and tautomers of guanidine-type groups, such as

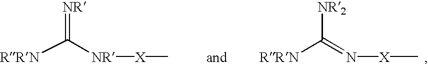

each tautomeric form is contemplated as being included within this invention whether existing in equilibrium or locked in one form by appropriate substitution with R′. The meaning of any substituent at any one occurrence is independent of its meaning, or any other substituent's meaning, at any other occurrence, unless specified otherwise.

The compounds of formula (I) inhibit the binding of vitronectin and other RGD-containing peptides to the vitronectin (αᵥβ₃) receptor. Inhibition of the vitronectin receptor on osteoclasts inhibits osteoclastic bone resorption and is useful in the treatment of diseases wherein bone resorption is associated with pathology, such as osteoporosis and osteoarthritis. Additionally, since the compounds of the instant invention inhibit vitronectin receptors on a number of different types of cells, said compounds would be useful in-the treatment of inflammatory disorders, such as rheumatoid arthritis and psoriasis, and cardiovascular diseases, such as atherosclerosis and restenosis. The compounds of Formula (I) of the present invention may be useful for the treatment or prevention of other diseases including, but not limited to, thromboembolic disorders, asthma, allergies, adult respiratory distress syndrome, graft versus host disease, organ transplant rejection, septic shock, eczema, contact dermatitis, inflammatory bowel disease, and other autoimmune diseases. The compounds of the present invention may also be useful for wound healing.

In particular, the compounds of the present invention are useful for the treatment, including prevention, of angiogenic disorders. The term angiogenic disorders as used herein includes conditions involving abnormal neovascularization. Where the growth of new blood vessels is the cause of, or contributes to, the pathology associated with a disease, inhibition of angiogenisis will reduce the deleterious effects of the disease. An example of such a disease target is diabetic retinopathy. Where the growth of new blood vessels is required to support growth of a deleterious tissue, inhibition of angiogenisis will reduce the blood supply to the tissue and thereby contribute to reduction in tissue mass based on blood supply requirements. Examples include growth of tumors where neovascularization is a continual requirement in order that the tumor grow and the establishment of solid tumor metastases. Thus, the compounds of the present invention inhibit tumor tissue angiogenesis, thereby preventing tumor metastasis and tumor growth.

Thus, according to the methods of the present invention, the inhibition of angiogenesis using the compounds of the present invention can ameliorate the symptoms of the disease, and, in some cases, can cure the disease.

A preferred therapeutic target for the compounds of the instant invention are eye diseases chacterized by neovascularization. Such eye diseases include corneal neovascular disorders, such as corneal transplantation, herpetic keratitis, luetic keratitis, pterygium and neovascular pannus associated with contact lens use. Additional eye diseases also include age-related macular degeneration, presumed ocular histoplasmosis, retinopathy of prematurity and neovascular glaucoma.

In another aspect of the invention is the use of the dual fibrinogen/vitronectin receptor antagonists of the present invention in the inhibition of platelet aggregation and smooth muscle cell migration following vascular injury from percutaneous transluminal coronary angioplasty (PTCA). The instant compounds are useful in vascular remodeling.

With reference to formula (Ia), preferably, Y is C(O).

With reference to formula (Ia), suitably, X¹ is CH₂, X² is C═O and A is NR′.

Preferably, with reference to formula (Ia):

X¹ is CH₂, X² is C═O, A is NR′, R′ is H or C₁₋₄alkyl, Y is C(O), R¹ is D-C₀₋₄alkyl, optionally substituted by R¹¹, and R² is —CH₂—CO₂H. In particular, R¹ is H, CH₃, CH₂CH₃, CH₂CF₃, (CH₂)₁₋₂phenyl, in which the phenyl group is unsubstituted or substituted by C₁₋₄alkyl, C₁₋₄alkoxy, C₁₋₄alkthio, CF₃, OH, or halo.

Suitable substituents for R⁵ when fibrinogen antagonist acitivity is desired are:

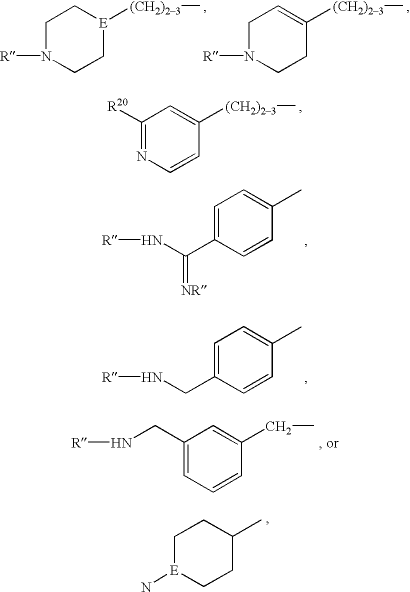

R″HNC(═NH)NH—(CH₂)₃(CHR¹⁰)—, and R″HN—(CH₂)₅—, wherein E is N or CH, and R²⁰ is hydrogen, amino, mono or di—C₁₋₄alkylamino, hydroxy or C₁₋₄alkyl.

Particularly good substituents for promoting fibrinogen antagonist activity are:

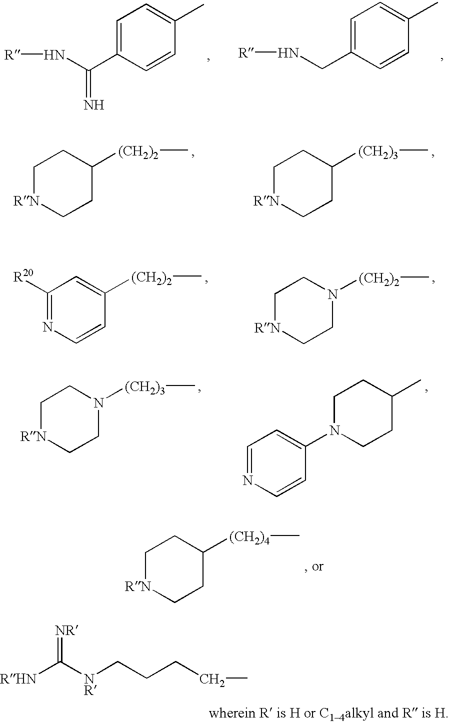

Particularly preferred of such groups for R⁵ are:

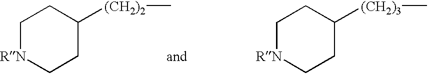

wherein R″ is H.

Preferred substituents for the group attached to W in R⁴ when vitronectin binding activity is desired are:

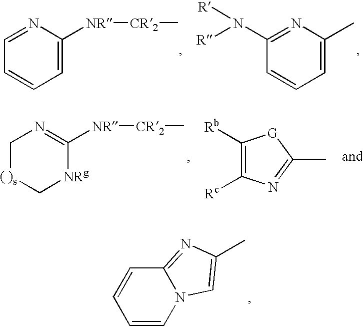

wherein G is NH. Preferably, R^{b} and R^{c} are joined to formn a cyclohexyl, phenyl or pyridyl ring. Suitably, R′, R″ and R^{g} are each H and s is 0 or 1.

Specific preferred R⁴ substituents for enhancing vitronectin activity are

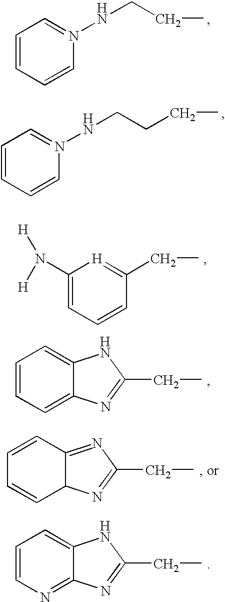

A particularly preferred of such groups for R⁴ is:

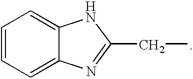

By appropriate selection of the spacing of the substituents R⁴ and R⁵ from the phenyl ring of the 6-7 ring system, compounds having selective activity for both the vitronectin and fibrinogen receptors, i.e. dual activity for both receptors, may be obtained. In general, fibrinogen antagonist activity will be favored by an intramolecular distance of about 16 intervening covalent bonds via the shortest path between the oxygen of the carbonyl moiety of the R² group attached to the seven-membered ring, and the basic nitrogen moiety of R⁵; while vitronectin antagonist activity will be favored by about 14 intervening covalent bonds via the shortest path between the oxygen of the carbonyl moiety attached to the seven-membered ring, and the basic nitrogen moiety of R⁴.

Specific compounds of this invention are:

(S)-7-[[N-(4-pyridinylpropyl)-N-(1H-benzimidazol-2-ylmethyl)]-aminocarbonyl]-2,3,4,5-tetrahydro-4-methyl-3-oxo-1H-1,4-benzodiazepine-2-acetic acid;

(S)-7-[[N-4-piperidinylpropyl-N-(1H-benzimidazol-2-ylmethyl)]-aminocarbonyl]-2,3,4,5-tetrahydro-4-methyl-3-oxo-1H-1,4-benzodiazepine-2-acetic acid;

(S)-7-[[N-4-piperidinylethyl-N-(1H-benzimidazol-2-ylmethyl)]-aminocarbonyl]-2,3,4,5-tetrahydro-4-methyl-3-oxo-1H-1,4-benzodiazepine-2-acetic acid; and

(+/−)-2,3,4,5-tetrahydro-3-oxo-8-[[N-4-piperidinylpropyl-N-(1H-benzimidazol-2-ylmethyl ) ]aminocarbonyl]-2-methyl-1H-2-benzazepine-4-acetic acid;

or a pharmaceutically acceptable salt thereof.

Abbreviations and symbols commonly used in the peptide and chemical arts are used herein to describe the compounds of this invention.

C₁₋₄alkyl as applied herein is meant to include methyl, ethyl, n-propyl, isopropyl, n-butyl, isobutyl and t-butyl. C₁₋₆alkyl additionally includes pentyl, n-pentyl, isopentyl, neopentyl and hexyl and the simple aliphatic isomers thereof. Any C₁₋₄alkyl or C₁₋₆alkyl group may be optionally substituted by R¹¹ unless otherwise indicated. C₀₋₄alkyl and C₀₋₆alkyl additionally indicates that no alkyl group need be present (e.g., that a covalent bond is present).

C₂₋₆ alkenyl as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene. 2-butene, isobutene and the several isomeric pentenes and hexenes. Both cis and trans isomers are included. Any C₂₋₆alkenyl group may be optionally substituted by R¹¹ unless otherwise indicated.

C₂₋₆ alkynyl means an alkyl group of 2 to 6 carbons wherein one carbon-carbon single bond is replaced by a carbon-carbon triple bond. C₂₋₆ alkynyl includes acetylene, 1-propyne. 2-propyne, 1-butyne, 2-butyne, 3-butyne and the simple isomers of pentyne and hexyne. Any sp³ carbon atom in the C₂₋₆alkynyl group may be optionally substituted by R¹¹.

C₁₋₄oxoalkyl refers to an alkyl group of up to four carbons wherein a CH₂ group is replaced by a C(O). or carbonyl, group. Substituted formyl, acetyl, 1-propanal, 2-propanone, 3-propanal, 2-butanone, 3-butanone, 1- and 4-butanal groups are representative. C₁₋₆oxoalkyl includes additionally the higher analogues and isomers of five and six carbons substituted by a carbonyl group. C₃₋₆oxoalkenyl and C₃₋₆oxoalkynyl refers to a C₃₋₆alkenyl or C₃₋₆alkynyl group wherein a CH₂ group is replaced by C(O) group. C₃₋₄oxoalkenyl includes 1-oxo-2-propenyl, 3-oxo-1-propenyl, 2-oxo-3-butenyl and the like.

A substituent on a C₁₋₆ alkyl, C₂₋₆ alkenyl, C₂₋₆ alkynyl or C₁₋₆ oxoalkyl group, such as R¹¹, may be on any carbon atom which results in a stable structure, and is available by conventional synthetic techniques.

D-C₁₋₆ alkyl refers to a C₁₋₆ alkyl group wherein in any position a carbon-hydrogen bond is replaced by a carbon-D bond. D-C₂₋₆ alkenyl and D-C₂₋₆ alkynyl have a similar meaning with respect to C₂₋₆ alkenyl and C₂₋₆ alkynyl.

Ar, or aryl, as applied herein, means phenyl or naphthyl, or phenyl or naphthyl substituted by one to three moieties R¹¹. In particular, R¹¹ may be C₁₋₄alkyl, C₁₋₄alkoxy, C₁₋₄alkthio, CF₃, OH, or halo.

Het, or heterocycle, indicates an optionally substituted five or six membered monocyclic ring, or a nine or ten-membered bicyclic ring containing one to three heteroatoms chosen from the group of nitrogen, oxygen and sulfur, which are stable and available by conventional chemical synthesis. Illustrative heterocycles are benzofuran, benzimidazole, benzopyran, benzothiophene, furan, imidazole, indole, indoline, morpholine, piperidine, piperazine, pyrrole, pyrrolidine, tetrahydropyridine, pyridine, thiazole, thiophene, quinoline, isoquinoline, and tetra- and perhydro- quinoline and isoquinoline. A six membered ring heterocycle containing one or two nitrogens, such as piperidine, piperazine, tetrahydropyridine and pyridine, are preferred heterocycles for the moiety Z. Any accessible combinatioln of up to three substituents, such as chosen from R¹¹, on the Het ring that is available by chemical synthesis and is stable is within the scope of this invention.

C₃₋₇cycloalkyl refers to an optionally substituted carbocyclic system of three to seven carbon atoms, which may contain up to two unsaturated carbon-carbon bonds. Typical of C₃₋₇cycloalkyl are cyclopropyl, cyclobutyl, cyclopentyl, cyclopentenyl, cyclohexyl, cyclohexenyl and cycloheptyl. Any combination of up to three substituents, such as chosen from R¹¹, on the cycloalkyl ring that is available by conventional chemical synthesis and is stable, is within the scope of this invention.

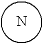

as used herein indicates a nitrogen heterocycle, which may be a saturated or unsaturated stable five-, six- or seven-membered monocyclic ring, or a seven- to ten-membered bicyclic ring containing up to three nitrogen atoms or containing one nitrogen atom and a hetcroatom chosen from oxygen and sulfur, and which may be substituted on any atom that results in a stable structure. The nitrogen atom in such ring may be substituted so as to result in a quaternary nitrogen. The nitrogen heterocycle may be substituted in any stable position by R²¹, for instance H, C₁₋₄alkoxy, F, Cl, Br, I, NO₂, NR′₂, OH, CO₂R′, CONHR′, CF₃, D-C₀₋₄alkyl, D-C₁₋₄alkyl-S(O)ᵤ (e.g., where u is 0, 1 or 2) or C₁₋₄alkyl substituted by any of the aforementioned sustituents. Representative of

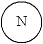

are pyrroline, pyrrolidine, imidazole, imidazoline, imidazolidine, pyrazole, pyrazoline, pyrazolidine, piperidine, piperazine, morpholine, pyridine, pyridinium, tetrahydropyridine, tetrahydro- and hexahydro-azepine, quinuclidine, quinuclidinium, quinoline, isoquinoline, and tetra- and perhydro- quinoline and isoquinoline. In particular,

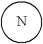

may be pyridyl, pyrolidinyl, piperidinyl, piperazinyl, azetidinyl, quinuclidinyl or tetrahydropyridinyl.

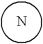

is preferably 4-pyridyl, 4-(2-amino-pyridyl), 4-tetrahydropyridyl, 4-piperidinyl or 4-piperazinyl.

When R^{b} and R^{c} are joined together to form a five- or six-membered aromatic or non-aromatic ring fused to the ring to which R^{b} and R^{c} are attached, the ring formed will generally be a five- or six-membered heterocycle selected from those listed above for Het, or will be a phenyl, cyclohexyl or cyclopentyl ring. Benzimidazolyl, 4-azabenzimidazolyl, 5-azabenzimidazolyl and substituted derivatives thereof are preferred moieties for the group attached to W in R⁴.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical. Cbz refers to the benzyloxycarbonyl radical, BrZ refers to the o-bromobenzyloxycarbonyl radical, ClZ refers to the o-chlorobenzyloxycarbonyl radical, Bn refers to the benzyl radical, 4-MBzl refers to the 4-methyl benzyl radical, Me refers to methyl, Et refers to ethyl, Ac refers to acetyl. Alk refers to C₁₋₄alkyl, Nph refers to 1- or 2-naphthyl and cHex refers to cyclohexyl. MeArg is N^{α}-methyl arginine. Tet refers to 5-tetrazolyl.

Certain reagents are abbreviated herein. DCC refers to dicyclohexylcarbodiimide, DMAP refers to dimethylaminopyridine, DIEA refers to diisopropylethylamine, EDC refers to N-ethyl-N′(dimethylaminopropyl)-carbodiimide. HOBt refers to 1-hydroxybenzotriazole, THF refers to tetrahydrofuran, DMF refers to dimethyl formamide. NBS refers to N-bromo-succinimide. Pd/C refers to a palladium on carbon catalyst. DPPA refers to diphenylphosphoryl azide, BOP refers to benzotriazol-1-yloxy-tris(dimethyiamino)phosphonium hexafluorophosphate, HF refers to hydrofluoric acid, PPA refers to polyphosphoric acid, TEA refers to triethylamine, TFA refers to trifluoroacetic acid, PCC refers to pyridinium chlorochromate. incorporated herein by reference.

Compounds of formula (I) are prepared by the methods described in Schemes I and II, and methods analogous to those described therein.

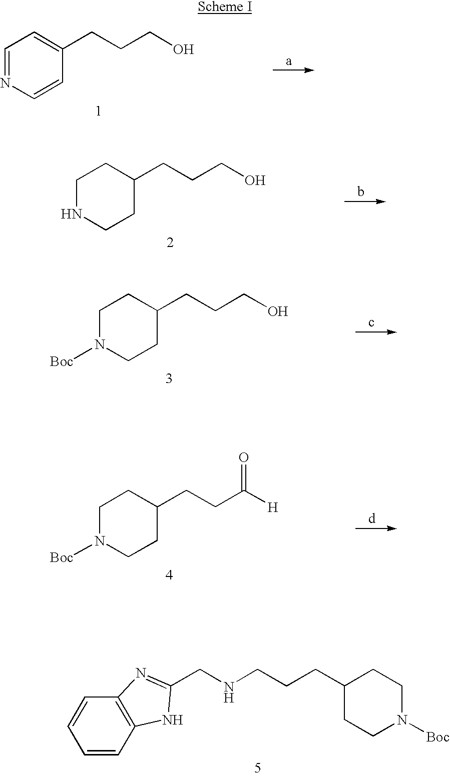

a) H₂, Pt₂O, 1N HCl, b) (Boc)₂O, 1N NaOH, dioxane: c) oxalyl chloride, DMSO, Et₃N, CH₂Cl₂; d) aminomethylbenzimidazole dihydrochloride, KOH, NaBH₃CN, MeOH.

An appropriately substituted pyridyl alcohol, such as I-1 (available from Aldrich Chemical Co., Milwaukee, Wis.), is reduced under catalytic hydrogenation conditions with platinum oxide in aqueous HCl to give the corresponding piperidinyl alcohol, such as I-2. Other useful methods to achieve this type of transformation can be found in Freifelder, “Practical Catalytic Hydrogenation”, Chapter 9 (published by Wiley-Interscience). The nitrogen is protected as the t-butyl carbamate I-3 by reaction with di-t-butyl dicarbonate in a mixture of aqueous NaOH and dioxane. The primary alcohol is oxidized to the corresponding aldehyde I-4 following the general procedure of Swern (J. Org. Chem. 1976, 41, 3329). Many alternative methods of oxidizing a primary alcohol to the corresponding aldehyde have been described and can be found in such reference volumes as “Compendium of Organic Synthetic Methods” (published by Wiley-Interscience). The reductive amination of I-4 with aminomethylbenzimidazole (available from Aldrich Chemical Co., Milwaukee, Wis.) is carried out following the general procedure of Borch (Org. Syn. 1972, 52, 124) to give the protected arginine mimic I-5.

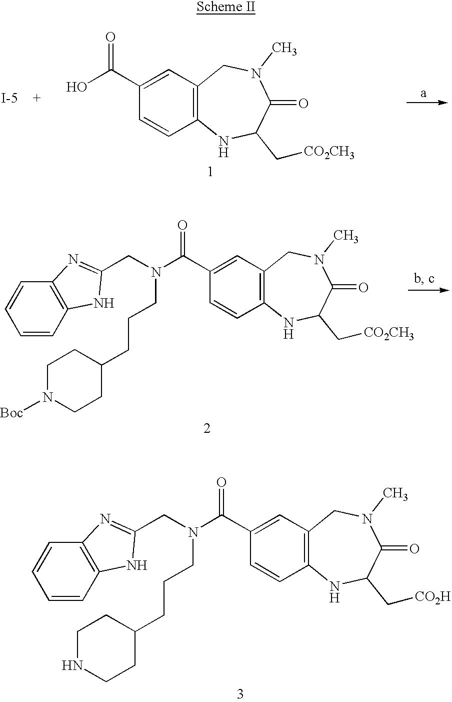

a) EDC, HOBT.H₂O, Et₃N, DMF; b) 1N NaOH, MeOH; c) TFA, CH₂Cl₂.

A suitably protected benzazepine or benzodiazepine, such as II-1 (prepared as described in Bondinell, et al., PCT application WO 93/00095, published Jan. 7, 1993 and Bondinell, et al., PCT application WO 94/14776, published Jul. 7, 1994) is converted to an activated form using, for example, EDC and HOBt, and the activated form is subsequently reacted with a suitably protected arginine mimic, such as I-5 (prepared as above), in a suitable solvent such as DMF or CH₂Cl₂. Depending on whether acid nuetralization is required, an added base, such as diisopropylethylamine, may be used. The methyl ester of II-2 is hydrolyzed with aqueous base, such as, NaOH in aqueous MeOH and the t-butyl carbarnate is removed with a strong acid, such as TFA in CH₂Cl₂ or HCl in dioxane. The intermediate carboxylate salt is acidified with a suitable acid, such as TFA or HCl to afford the carboxylic acid II-3.

The tricyclic core of the compounds of formula (II) are prepared as described in Bondinell, et al., PCT Application WO 97/01540, published Jan. 16, 1997. Reference should be made to said patent application. the entire disclosure of which is incorporated by reference. The formula (II) compounds are prepared by methods analogous to those detailed in Scheme II.

Coupling methods to form amide bonds are generally well known to the art. The methods of peptide synthesis generally set forth by Bodansky et al.. THE PRACTICE OF PEPTIDE SYNTHESIS, Springer-Verlag, Berlin. 1984, Ali et al. in J. Med. Chem., 29, 984 (1986) and J. Med. Chem., 30, 2291 (1987) are generally illustrative of the technique and are incorporated herein by reference. Coupling reagents as used herein denote reagents which may be used to form amide bonds. Typical coupling methods employ carbodiimides, activated anhydrides and esters and acyl halides. Reagents such as EDC, DCC, DPPA, BGP reagent, HOBt, N-hydroxysuccinimide and oxalyl chloride are typical.

Typically, the amine or aniline is coupled via its free amino group to an appropriate carboxylic acid substrate using a suitable carbodiumide coupling agent. such as N,N′ dicyclohexyl carbodumide (DCC), optionally in the presence of catalysts such as 1-hydroxybenzotriazole (HOBt) and dimethylamino pyridine (DMAP) Other methods, such as the formation of activated esters, anhydrides or acid halides, of the free carboxyl of a suitably protected acid substrate, and subsequent reaction with the free amine of a suitably protected amine, optionally in the presence of a base, are also suitable. For example, a protected Boc-amino acid or Cbz-amidino benzoic acid is treated in an anhydrous solvent, such as methylene chloride or tetrahydrofuran (THF), in the presence of a base, such as N-methyl morpholinc. DMAP or a trialkylamine, with isobutyl chloroformate to form the “activated anhydridc”, which is subsequently reacted with the free amine of a second protected amino acid or aniline.

Acid addition salts of the compounds are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable, Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts.

This invention also provides a pharmaceutical composition which comprises a compound according to formula (I) and a pharmaceutically acceptable carrier. Accordingly, the compounds of formula (I) may be used in the manufacture of a medicament. Pharmaceutical compositions of the compounds of formula (I) prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in a emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or nonaqueous suspension. Such a liquid formulation may be administered directly p.o. or filled into a soft gelatin capsule.

For rectal administration, the compounds of this invention may also be combined with excipients such as cocoa butter. glycerin, gelatin or polyethylene glycols and molded into a suppository.

The compounds described herein which are antagonists of the vitronectin receptor, are useful for treating diseases wherein the underlying pathology is attributable to ligand or cell which interacts with the vitronectin receptor. For instance, these compounds are useful for the treatment of diseases wherein loss of the bone matrix creates pathology. Thus, the instant compounds are useful for the treatment of ostoeporosis, hyperparathyroidism, Paget's disease, hypercalcemia of malignancy, osteolytic lesions produced by bone metastasis, bone loss due to immobilization or sex hormone deficiency. The compounds of this invention are also believed to have utility as antitumor, antiinflammatory, anti-angiogenic and anti-metastatic agents, and be useful in the treatment of cancer, atherosclerosis and restenosis. In particular, the compounds of this invention are useful for inhibiting restenosis following angioplasty.

The compounds of this invention which inhibit fibrinogen binding provide a method of inhibiting platelet aggregation and clot formation in a mammal, especially a human, which comprises the internal administration of a compound of formula (I) and a pharmaceutically acceptable carrier. Indications for such therapy include acute myocardial infarction (AMI), deep vein thrombosis, pulmonary embolism, dissecting anurysm, transient ischenua attack (TIA), stroke and other infarct-related disorders, and unstable angina. Chronic or acute states of hyper-aggregability, such as disseminated intravascular coagulation (DIC), septicemia, surgical or infectious shock, post-operative and postpartum trauma, cardiopulmonary bypass surgery, incompatible blood transfusion, abruptio placenta, thrombotic thrombocytopenic purpura (TTP), snake venom and immune diseases, are likely to be responsive to such treatment. In addition, the compounds of this invention may be useful in a method for the prevention of metastatic conditions, the prevention or treatment of fungal or bacterial infection, inducing immunostimulation, treatment of sickle cell disease, and the prevention or treatment of diseases in which bone resorption is a factor.

This invention further provides a method for inhibiting the reocclusion of an artery or vein following fibrinolytic therapy, which comprises internal administration of a compound of formula (I) and a fibrinolytic agent. Administration of a compound of formula (I) in fibrinolytic therapy either prevents reocclusion completely or prolongs the time to reocclusion. When used in the context of this invention the term fibrinolytic agent is intended to mean any compound, whether a natural or synthetic product, which directly or indirectly causes the lysis of a fibrin clot. Plasminogen activators are a well known group of fibrinolytic agents. Useful plasminogen activators include, for example. anistreplase, urokinase (UK), pro-urokinase (pUK), streptokinase (SK), tissue plasminogen activator (tPA) and mutants, or variants, thereof.

The compounds of this invention may also be used in vitro to inhibit the aggregation of platelets in blood and blood products, e.g., for storage, or for ex vivo manipulations such as in diagnostic or research use.

The compound is administered either orally or parenterally to the patient, in a manner such that the concentration of drug is sufficient to inhibit bone resorption, or inhibit platelet aggregation or other such indication. The pharmaceutical composition containing the compound is administered at an oral dose of between about 0.1 to about 50 mg/kg in a manner consistent with the condition of the patient. Preferably the oral dose would be about 0.5 to about 20 mg/kg. For acute therapy, parenteral administration is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg. The compounds are administered one to four times dally at a level to achieve a total daily dose of about 0.4 to about 400 mg/kg/day. The precise level and method by which the compounds are administered is readily determined by one routinely skilled in the art by comparing the blood level of the agent to the concentration required to have a therapeutic effect.

The compounds may be tested in one of several biological assays to determine the concentration of compound which is required to have a given pharmacological effect.

### INHIBITION OF VITRONECTIN BINDING

Solid-Phase [³H]-SK&F-107260 Binding to αᵥβ₃: Human placenta or human platelet αᵥβ₃ (0.1-0.3 mg/mL) in buffer T (containing 2 mM CaCl₂ and 1% octyllucoside) was diluted with buffer T containing 1 mM CaCl₂, 1 mM MnCl₂, 1 mM MgCl₂ (buffer A) and 0.05% NaN₃, and then immediately added to 96-well ELISA plates (Corning, New York. N.Y.) at 0.1 mL per well. 0.1 - 0.2 μg of αᵥβ₃ was added per well. The plates were incubated overnight at 4° C. At the time of the experiment, the wells were washed once with buffer A and were incubated with 0.1 mL of 3.5% bovine serum albumin in the same buffer for 1 hr at room temperature. Following incubation the wells were aspirated completely and washed twice with 0.2 mL buffer A.

Compounds were dissolved in 100% DMSO to give a 2 mM stock solution, which was diluted with binding buffer (15 mM Tris-HCl (pH 7.4), 100 mM NaCl, 1 mM CaCl₂, 1 nmM MnCl₂, 1 mM MgCl₂) to a final compound concentration of 100 μM. This solution is then diluted to the required final compound concentration. Various concentrations of unlabeled antagonists (0.001 - 100 μM) were added to the wells in triplicates, followed by the addition of 5.0 nM of [³H]-SK&F-107260 (65 -86 Ci/mmol).

The plates were incubated for 1 hr at room temperature. Following incubation the wells were aspirated completely and washed once with 0.2 mL of ice cold buffer A in a well-to-well fashion. The receptors were solubilized with 0.1 mL of 1% SDS and the bound [³H-SK&F-107260 was determined by liquid scintillation counting with the addition of 3 mL Ready Safe in a Beckman LS Liquid Scintillation Counter, with 40% efficiency. Nonspecific binding of [³H]-SK&F-107260 was determined in the presence of 2 μM SK&F-107260 and was consistently less than 1% of total radioligand input. The IC₅₀ (concentration of the antagonist to inhibit 50% binding of [³H]-SK&F-107260) was determined by a nonlinear, least squares curve-fitting routine, which was modified from the LUNDON-2 program. The K₁ (dissociation constant of the antagonist) was calculated according to the equation: K₁=IC₅₀/(1+L/K_{d}), where L and K_{d} were the concentration and the dissociation constant of [³H]-SK&F-107260. respectively.

Compounds of the present invention inhibit vitronectin binding to SK&F 107260 in the concentration range of about 4 to about 25 nM

### INHIBITION OF RGD-MEDIATED α_{IIb}β₃ BINDING

Purification of α_{IIb}β₃

Ten units of outdated. washed human platelets (obtained from Red Cross) were lyzed by gentle stirring in 3% octylglucoside, 20 mM Tris-HCl, pH 7.4, 140 mM NaCl, 2 mM CaCl₂ at 4° C. for 2 h. The lysate was centrifuged at 100,000 g for 1 h. The supernatant obtained was applied to a 5 mL lentil lectin sepharose 4B column (E. Y. Labs) preequilibrated with 20 mM Tris-HCl, pH 7.4, 100 mM NaCl, 2 mM CaCl₂. 1% octylglucoside (buffer A). After 2 h incubation, the column was washed with 50 mL cold buffer A. The lectin-retained α_{IIb}β₃ was eluted with buffer A containing 10% dextrose. All procedures were performed at 4° C. The α_{IIb}β₃ obtained was >95% pure as shown by SDS polyacrylamide gel electrophoresis.

Incorporaltion of α_{IIb}β₃ Liposomes

A mixture of phosphatidylserine (70%) and phosphatidylcholine (30%) (Avanti Polar Lipids) were dried to the walls of a glass tube under a stream of nitrogen. Purified α_{IIb}β₃ was diluted to a final concentration of 0.5 mg/mL and mixed with the phospholipids in a protein:phospholipid ratio of 1:3 (w:w). The mixture was resuspended and sonicated in a bath sonicator for 5 min. The mixture was then dialyzed overnight using 12.000-14.000 molecular weight cutoff dialysis tubing against a 1000-fold excess of 50 mM Tris-HCl, pH 7.4, 100 mM NaCl, 2 mM CaCl2 (with 2 changes). The α_{IIb}β₃-containing liposomes wee centrifuged at 12,000 g for 15 min and resuspended in the dialysis buffer at a final protein concentration of approximately 1 mg/mL. The liposomes were stored at −70° C. until needed.

Competitive Binding to α_{IIb}β₃

The binding to the fibrinogen receptor (α_{IIb}β₃) was assayed by an indirect competitive binding method using [³H]-SK&F-107260 as an RGD-type ligand. The binding assay was performed in a 96-well filtration plate assembly (Millipore Corporation, Bedford, Mass.) using 0.22 um hydrophilic durapore membranes. The wells were precoated with 0.2 mL of 10 μg/mL polylysine (Sigma Chemical Co., St. Louis, Mo.) at room temperature for 1 h to block nonspecific binding. Various concentrations of unlabeled benzadiazapines were added to the wells in quadruplicate. [³H]-SK&F-107260 was applied to each well at a final concentration of 4.5 nM, followed by the addition of 1 μg of the purified platelet α_{IIb}β₃-containing liposomes. The mixtures were incubated for 1 h at room temperature. The α_{IIb}β₃-bound [3H]-SK&F-107260 was seperated from the unbound by filtration using a Millipore filtration manifold, followed by washing with ice-cold buffer (2 times, each 0.2 mL). Bound radioactivity remaining on the filters was counted in 1.5 mL Ready Solve (Beckman Instruments, Fullerton, Calif.) in a Beckman Liquid Scintillation Counter (Model LS6800), with 40% efficiency. Nonspecific binding was determined in the presence of 2 μM unlabeled SK&F-107260 and was consistently less than 0.14% of the total radioactivity added to the samples. All data points are the mean of quadruplicate determinations.

Competition binding data were analyzed by a nonlinear least-squares curve fitting procedure. This method provides the IC50 of the antagonists (concentration of the antagonist which inhibits specific binding of [³H]-SK&F-107260 by 50% at equilibrium). The IC50 is related to the equilibrium dissociation constant (Ki) of the antagonist based on the Cheng and Prusoff equation: Ki=IC50/(1+L/Kd), where L is the concentration of [3H]-SK&F-107260 used in the competitive binding assay (4.5 nM), and Kd is the dissociation constant of [3H]-SK&F-107260 which is 4.5 nM as determined by Scatchard analysis.

Compounds of this invention inhibit [³H]-SK&F-107260 binding with a Ki of about 8 to about 55 nM.

Inhibition of platelet aggregation may be measured by the method described in WO 93/00095 (PCT/US/92/05463). In vivo thrombus formation is demonstrated by recording the systemic and hemodynaniic effects of infusion of the peptides into anesthetized dogs according to the methods described in Aiken et al., Prostaglandins, 19, 620 (1980).

Vascular Smooth Muscle Cell Migration Assay

The compounds of the instant invention were tested for their ability to inhibit the migration and proliferation of smooth muscle tissue in an artery or vein in order to assess their ability to prevent restenosis of an artery, such as that which typically occurs following angioplasty.

Rat or human aortic smooth muscle cells were used. The cell migration was monitored in a Transwell cell culture chamber by using a polycarbonate membrane with pores of 8 um (Costar). The lower surface of the filter was coated with vitronectin. Cells were suspended in DMEM supplemented with 0.2% bovine serum albumin at a concentration of 2.5−5.0×10⁶ cells/mL, and were pretreated with test compound at various concentrations for 20 min at 20° C. The solvent alone was used as control. 0.2 mL of the cell suspension was placed in the upper compartment of the chamber. The lower compartment contained 0.6 mL of DMEM supplemented with 0.2% bovine serum albumin. Incubation was carried out at 37° C. in an atmosphere of 95% air/5% CO₂ for 24 hr. After incubation, the non-migrated cells on the upper surface of the filter were removed by gentle scraping. The filter was then fixed in methanol and stained with 10% Giemsa stain. Migration was measured either by a) counting the number of cells that had migrated to the lower surface of the filter or by b) extracting the stained cells with 10% acetic acid followed by determining the absorbance at 600 nM.

The efficacy of the compounds of formula (I) to prevent tumor growth may be determined using several transplantable mouse tumor models. See U.S. Pat. Nos. 5,004,758 and 5,633,016 for details of these models.

The examples which follow are intended to in no way limit the scope of this invention, but are provided to illustrate how to make and use the compound of this invention. Many other embodiments will be readily apparent and available to those skilled in the art.

### General

Nuclear magnetic resonance spectra were obtained using either a Bruker A M 250 or Bruker AC 400 spectrometer. Chemical shifts are reported in parts per milliom (δ) downfield from the internal standard tetramethylsilane. Mass spectra were taken on either VG 70 FE or VG ZAB HF instruments using fast atom bombardment (FAB) or electrospry-(ES) ionization techniques. Elemental analyses were performed by Quantitative Technologies Inc., Whitehouse, N.J.

Analtech Silica Gel GF and E. Merck Silica Gel 60 F-254 thin layer plates were used for thin layer chromatography. Flash chromatography was carried out on E. Merck Kieselgel 60 (230-400 mesh) silica gel. Analytical and preparative HPLC were carried out on Bechman Chromatographs. PRP-1® is a polymeric (styrene-divinylbenzene) chromatographic support, and is a registered trademark of Hamilton Co.. Reno, Nev.

### EXAMPLE 1

### Preparation of (S)-7-[[N-4-piperidinylethyl-N-(1H-benzimidazol-2-ylmethyl)]aminocarbonyl]-2,3,4,5-tetrahydro-4-methyl-3-oxo-1H-1,4-benzodiazepine-2-acetic Acid

a) N-t-Butoxycarbonyl-4-(2-hydroxyethyl)piperidine

To a solution of 4-(2-hydroxyethyl)pyridine (1.00 g, 8.12 mmol) in 1N HCl (10 mL) in a Parr hydrogenation apparatus was added Pt₂O (10 mg). The mixture was hydrogcnated at 50 psi for 7 h. After venting the hydrogen, the catalyst was removed by filtration through celite and the celite rinsed with H₂O (10 mL). Concentration of the combined filtrate afforded the crude 4-(2-hydroyethyl)piperidine MS (ES+) m/z 129.9 (M+H⁺).

The crude material obtained above was dissolved in a mixture of dioxane (25 mL) and 1N NaOH (25 mL). The resulting solution was cooled to 0° C. and di-t-butyl dicarbonate (2.12 g. 9.71 mmol) was added. The reaction was allowed to warm to RT and after stirring overnight was extracted with EtOAc (3×50 mL). The combined organic extracts were washed with brine (50 mL) and dried over Na₂SO₄. Concentration gave a pale yellow oil (1.56 g) which was subjected to radial chromatography (solvent gradient 30% to 50% EtOAc/hexane, silica gel, 6 mm plate) to give 1.38 g (74%) of the desired material as a clear oil. 1H NMR (400 MHz, CDCl₃) δ 4.10 (broad s, 2H), 3.70 (dd, J=11.8, 6.4 Hz, 2H), 2.58 (m, 2H), 1.70

b) 2-(4-N-t-Butoxycarbonylpiperidine)acetaldehyde

To a solution of oxalyl chloride (0.53 mL, 6.07 mmol) and DMSO (0.86 mL, 12.1 mmol) in CH₂Cl₂ (15 mL) at −78° C. was added rapidly a solution of N-t-butoxycarbonyl-4-(2-hydroxyethyl)piperidine (1.38 g, 6.03 mmol). The reaction was warmed to −15° C. for 30 min. Et₃N (1.68 mL, 12.o mmol) was added to this solution and the reaction was allowed to warm and maintained at RT for 3 h. The reaction was quenched by pouring into sat. NaHCO₃ (10 mL) and extracting with CH₂Cl₂ (3×15 mL). The combined organic extracts were dried over Na₂SO₄ and concentrated to give a pale green oil (1.42 g). Purification by radial chromatography (50% EtOAc/hexanes, silica gel, 6 mm plate) gave 1.08 g (79%) of the desired product as a pale yellow oil. 1 H NMR (400 MHz, CDCl₃) δ 9.79 (s, 1H), 4.10 (m, 2H), 2.70 (m, 2H0, 2.38 (d, J=6.7 Hz, 2H), 2.10 (m, 2H), 1.70 (mn, 2H), 1.45 (s, 9H), 1.15 (m, 2H).

c) N-[(4-N-t-Butoxycarbonylpiperidinylethyl)-N-(1H-benzimidazol-2-ylmethyl)]amine

To aminomethylbenzimidazole dihydrochloride (1.31 g, 5.95 mmol) in MeOH (8 mL) was added KOH (0.67 g, 11.9 mmol). When the KOH had dissolved, a solution of 2-(4-N-t-butoxycarbonylpiperidine)acetaldehyde (1.08 g, 4.76 mmol) in MeOH (4 mL) was added. After 15 min at RT, NaCNBH₃ (0.10 g, 1.59 mmol) was added portionwise. The reaction was allowed to proceed for 8 h and then concentrated to give a yellow residue. Radial chromatography (10% MeOH/CHCl₃, silica gel, 6 mm plate) gave 0.76 g (45%) of the desried material as a pale yellow oil. MS (ES+) m/z 359.3 (M+H⁺).

d) (S)-7-[[N-(4-N-t-Butoxycarbonylpiperidinylethyl)-N-(1H-benzimidazol-2-ylmethyl)]aminocarbonyl]-2,3,4,5-tetrahydro-4-methyl-3-oxo-1H-1,4-benzodiazepine-2-acetic acid, methyl ester

N-[(4-N-t-Butoxycarbonylpiperidinylethyl )-N-(1H-benzimidazol-2-ylmethyl)]amine (0.66 g, 1.84 mmol), methyl (S)-7-carboxy-2,3,4,5-tetrahydro-3-oxo-4-methyl-1H-1,4-benzodiazepine-2-acetate (0.54 g 1.85 mmol), EDC (0.53 g, 2.76 mmol), HOBT (0.38 g, 2.81 mmol) and Et₃N (1.30 mL, 9.33 mmol) were combined in DMF (20 mL) and stirred at RT for 3 days. The solvent was evaporated and the product was isolated by radial chromatography (5% MeOH/CHCl₃, silica gel, 6 mm plate) to give 1.06 g of material which was deemed to be of sufficient purity by 1H NMR to be carried onto the next step. MS (ES+) m/z 633.3 (M+H⁺).

e) (S)-7-[[N-4-Piperidinylethyl-N-(-1H-benzimidazol-2-ylmethyl)]aminocarbonyl]-2,3,4,5-tetrahydro-4-methyl-3-oxo-1H-1,4-benzodiazepine-2-acetic Acid

To (S)-7-[[N-(4-N-t-butoxycarbonylpiperidinylethyl)-N-(1H-benzimidazol-2-ylmethyl)]aminocarbonyl]-2,3,4,5-tetrahydro-4-methyl-3-oxo-1H-1,4-benzodiazepine-2-acetic acid, methyl ester (1.06 g, 1.68 mmol) in EtOH (5 mL) was added 1N NaOH (5 mL, 5 mmol). After stirring at RT for 4h, the reaction was made acidic to pH 3 with 1 N HCl and the solvent was evaporated. Azeotrope the residue from toluene to give the product acid as a pale yellow foam. This was carried on crude without further purification. MS (ES+) m/z 619.2 (M+H⁺).

The crude acid obtained above was dissolved in CH₂Cl₂ (10 mL) and TFA (10 mL). After 2h at RT, the solvent was removed under reduced pressure to give an off-white foam. Flash chromatography (reverse phase silica gel, step gradient: H₂O+0.1% TFA, 5% CH₃CN/H₂O+0.1% TFA, 10% CH₃CN/H₂O+0.1% TFA, 20% CH₃CN/H₂O+0.1% TFA) gave 0.38 g of desired product as a white fluffy solid which was pure by HPLC analysis. MS(ES+) m/z 519.4 (M+H⁺). Anal. (C₂₈H₃₄N₆O₄.2CF₃CO₂H.2H₂O) caicd: C, 49.11 H, 5.15; N, 10.74. Found: C, 49.26; H, 5.00; N, 10.59.

### EXAMPLE 2

### Preparation of (S)-7-[[N-4-piperidinylpropyl-N-(1-benzimidazol-2-ylmethyl)]aminocarbonyl]-2,3,4,5-tetrahydro-4-methyl-3-oxo-1H-1,4-benzodiazepine-2-acetic Acid

a) N-t-Butoxycarbonyl-4-(3-hydroxypropyl)piperidine

In a manner analogous to Example 1(a), 4-pyridinepropanol (2.21 g, 15.5 mmol) gave 3.97 g of the desired product which was used without further purification. ¹H NMR (250 MHz, CDCl₃) δ 4.10 (m, 2H), 3.60 (m, 2H), 2.65 (m, 2H), 1.70 - 1.00 (m, 9H), 1.45 (s, 9H).

b) 3-(4-N-t-Butoxycarbonylpiperidine)propanal

In a manner analogous to Example 1(b), N-t-butoxycarbonyl4-(3-hydroxypropyl)piperidine (1.00 g, 4.11 mmol) gave 0.68 g of the desired product as a clear oil after flash chromatography (20% to 50% EtOAc/hexanes, silica gel). ¹H NMR (250 MHz, CDCl₃) δ 9.78 (s, 1H), 4.09 (m, 2H), 2.66 (m, 2H), 2.47 (m, 2H), 1.70-1.35 (m, 5H), 1.45 (s, 9H), 1.10 (m, 2H).

c) N-[(4-N-t-Butoxycarbonylpiperidinylpropyl)-N-(1H-benzimidazol-2-ylmethyl)]amine

In a manner analogous to Example 1(c), 3-(4-N-t-butoxycarbonylpiperidine)-propanal (0.12 g, 0.50 mmol). aminomethylbenzimidazole dihydrochloride (0.14 g, 0.64 mmol), KOH (0.07 g, 1.25 mmol) and NaCNBH₃ (12.0 mg, 0.19 mmol) gave 0.05 g of the desired product as a pale yellow oil after flash chromatography (5% MeOH/CHCl₃, silica gel). MS (ES+) m/z 373.4 (M+H⁺).

d) (S)-7-[[N-(4-N-t-Butoxycarbonylpiperidinylpropyl)-N-(1H-benzimidazol-2-ylmethyl)]aminocarbonyl]-2,3,4,5-tetrahydro-4-methyl-3-oxo-1H-1,4-benzodiazepine-2-acetic acid, methyl ester.

In a manner analogous to Example 1(d), N-[(4-N-t-butoxycarbonylpiperidinyl-propyl)-N-(1H-benzimidazol-2-ylmethyl)]amine (0.05 g, 0.13 mmol), methyl (S)-7-carboxy-2,3,4,5-tetrahydro-3-oxo-4-methyl-1H-1,4-benzodiazepine-2-acetate (0.04 g. 0.13 mmol), EDC (0.04 g, 0.21 mmol), HOBT (0.03 g, 0.22 mmol) and Et₃N (0.09 mL, 0.64 mmol) gave 0.14 g of the desired material after flash chromatography (5% MeOH/CHCl₃, silica gel). MS (ES+) m/z 647.5 (M+H⁺).

e) (S)-7-[[N-4-Piperidinylpropyl-N-(1H-benzimidazol-2-ylmethyl)]aminocarbonyl]-2,3,4,5-tetrahydro-4-methyl-3-oxo-1H-1,4-benzodiaiepine-2-acetic Acid

In a manner analogous to Example 1(e), (S)-7-[[N-(4-N-t-butoxycarbonyl-piperidinylpropyl)-N-(1H-benzimidazol-2-ylmethyl)]aminocarbonyl]-2,3,4,5-tetrahydro-4-methyl-3-oxo-1H-1,4-benzodiazepine-2-acetic acid, methyl ester (0.12 g, 0.19 mmol) gave 63.5 mg of the desired product as white solid after flash chromatography (step gradient: H₂O+0.1% TFA; 5% CH₃CN/H₂O+0.1% TFA; 10% CH₃CN/H₂O+0.1% TFA, reverse-phase silica gel). MS (ES+) m/z 533.5 (M+H⁺). Anal. (C₂₉H₃₆N₆O₄.2CF₃CO₂H.2H₂O) calcd: C, 49.75; H, 5.31; N, 10.55. Found: C, 49.64; H, 4.92; N, 10.24.

### EXAMPLE 3

### Preparation of (S)-7-[[N-(4-piperidinylpropyl)-N-(1H-4,5,6,7-tetrahydrobenzimidazol-2-ylmethyl)]aminocarbonyl]-2,3,4,5-tetrahydro-4-methyl-3-oxo-1H-1,4-benzodiazepine-2-acetic Acid

a) 3-(4-Pyridine)propanal

In a manner analogous to Example 1(b), 4-pyridinepopanol (1.76 g, 12.8 mmol). DMSO (2.73 mL, 38.5 mmol), oxalyl chloride (1.68 mL. 19.3 mmol) and Et₃N (8.86 mL, 63.5 mmol) gave 1.66 g of the desired product as a dark residue which was used without further purification. MS (136.0 (M+H⁺).

b) N-[(4-Pyridinylpropyl )-N-(1H-benzimidazol-2-ylmethyl)]amine

In manner analogous to Example 1(c), 3-(4-pyridine)propanal (1.66 g, 12.3 mmol), aminomethylbenzimidazole dihydrochloride (3.38 g, 15.4 mmol), KOH (1.90 g. 33.9 mmol) and NaCNBH₃ (0.26 g, 4.14 mmol) gave 1.97 g of the desired product as a clear oil after flash chromatography (10% to 15% MeOH/EtOAc, silica gel). MS (ES+) m/z 267.4 (M+H⁺).

c) (S)-7-[[N-(4-Pyridinylpropyl)-N-(1H-benzimidazol-2-ylmethyl)]aminocarbonyl]-2,3,4,5-tetrahydro-4-methyl-3-oxo-1H-1,4-benzodiazepine-2-acetic acid, methyl ester

In a manner analogous to Example 1(d), N-[(4-pyridinylpropyl)-N-(1H-benzimidazol-2-ylmethyl)]amine (0.41 g, 1.54 mmol), methyl (S)-7-carboxy-2,3,4,5-tetrahydro-3-oxo-4-methyl-1H-1,4-benzodiazepine-2-acetate (0.38 g, 1.30 mmol), EDC (0.38 g. 1.98 mmol), HOBT (0.27 g, 1.99 mmol), and Et₂N (0.90 mL, 6.46 mmol) gave 0.73 of the desired compound as a clear oil after flash chromatography (10% MeOH/CHCl₃, silica gel). MS(ES+) m/z 541.5 (M+H⁺).

d) (S)-7-[[N-(4-Piperidinylpropyl )-N-(1H-4,5,6,7-tetrahydrobenzimidazol-2-ylmethyl)aminocarbonyl]-2,3,4,5-tetrahydro-4-methyl-3-oxo-1H-1,4-benzodiazepine-2-acetic acid, methyl ester

A Parr hydrogenation apparatus was charged with (S)-7-[[N-(4-pyridinylpropyl)-N-(1 H-benzimidazol-2-ylmethyl)]aminocarbonyl]-2,3,4,5-tetrahydro-4-methyl-3-oxo-1H-1,4-benzodiazepine-2-acetic acid, methyl ester (0.10 g, 0.18 mmol), 1N HCl (0.18 ml) and PtO₂ (10 mg) in MeOH (1 mL). The material was hydrogenated at 40 psi and RT for 8 h. After venting the hydrogen and removing the catalyst by filtration, the solvent was evaporated under reduced pressure to give 0.09 g of the desired material as a white solid. This was used in the next step without further purification. MS (ES+) m/z 551.5 (M+H⁺).

e) (S)-7-[[N-(4-Piperidinylpropyl)-N-(1H-4,5,6,7-tetrahydrobenzimidazol-2-ylmethyl)]aminocarbonyl]-2,3,4,5-tetrahydro-4-methyl-3-oxo-1H-1,4-benzodiazepine-2-acetic acid

To the crude (S)-7-[[N-(4-Piperldinylpropyl)-N-(1H-4,5,6,7-tetrahydrobenzimidazol-2-ylmethyl)]aminocarbonyl]-2,3,4,5-tetrahydro-4-methyl-3-oxo-1H-1,4-benzodiazepine-2-acetic acid, methyl ester (0.09 g, 0.16 mmol) in EtOH (3 mL) was added 1N NaOH (0.54 mL). After stirring at RT for 7 h, the reaction mixture was made acidic (pH=3) with 1N HCl and concentrated to give a white residue. Trituration with EtOH gave the product as a white precipitate which was collected and dried under vacuum to give 0.05 g of the desired product as a white solid. MS(ES+) m/z 537.5 (M+H⁺).

### EXAMPLE 4

### Preparation of (+/−)-2,3,4,5-tetrahydro-3-oxo-8-[[N-4-piperidinylpropyl-N-(1H-benzimidazol-2-ylmethyl)]aminocarbonyl]1-2-methyl-1H-2-benzazepine-4-acetic Acid

a) (+/−)-2,3,4,5-Tetrahydro-3-oxo-8-[[N-(4-N-t-butoxycarbonylpiperidinylpropyl-N-(1H-benzimidazol-2-ylmethyl)]aminocarbonyl]-2-methyl-1H-2-benzazepine-4-acetic acid, methyl ester

In a manner analogous to Example 1(d), N-[(4-N-t-butoxycarbonylpiperidinyl-propyl)-N-(1H-benzimidazol-2-ylmethyl)]amine (0.14 g, 0.38 mmol), (+/−)-methyl 2,3,4,5-tetrahydro-3-oxo-8-carboxy-2-methyl-1H-2-benzazepine-4-acetate (0.12 g, 0.41 mmol), EDC (0.11 g, 0.57 mmol), HOBT (0.08 g, 0.59 mmol) and Et₃N (0.26 mL, 1.87) gave 0.19 g of the desired product after radial chromatography (10% MeOH/EtOAc, silica gel, 2 mm plate). MS (ES+) m/z 646.5 (M+H⁺).

b) (+/−)-2,3,4,5-tetrahydro-3-oxo-8-[[N-4-piperidinylpropyl-N-(1H-benzimidazol-2-ylmethyl)]aminocarbonyl]-2-methyl-1H-2-benzazepine-4-acetic acid

In a manner analogous to Example 1(e), (+/−)-2,3,4,5-tetrahydro-3-oxo-8-[[N-(4-N-t-butoxycarbonylpiperidinylpropyl-N-(1H-benzimidazol-2-ylmethyl)]aminocarbonyl]-2-methyl-1H-2-benzazepine-4-acetic acid, methyl ester (0.19 g, 0.29 mmol) gave 0.23 g of the desired product as a white foam after reverse phase flash chromatography (step gradient: H₂O+0.1% TFA then 30% CH₃CN/H₂O+0.1% TFA, Analytichem Bond-Elut® C-8 column, 50 mL size). MS (ES+) m/z 532.4 (M+H⁺). Anal. (C₃₀H₃₇N₅O₄.2CF₃CO₂H.2H₂O) calcd: C, 51.32; H, 5.45; N, 8.80. Found: C, 51.24; H, 5.15; N, 8.47.

### EXAMPLE 5

### Oral Dosage Unit Composition

A tablet for oral administration is prepared by mixing and granulating 20 mg of sucrose, 150 mg of calcium sulfate dihydrate and 50 mg of the compound of Example 1 with a 10% gelatin solution. The wet granules are screened, dried, mixed with 10 mg starch. 5 mg talc and 3 mg stearic acid; and compressed into a tablet.

The above description fully discloses how to make and use the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

What is claimed is:

1. A compound according to formulae (I) or (II): [chemical structure]
wherein:
A is NR′, CHR′, O or S;
A₁ is C or N;
X¹ and X² are C═O or CHR′, with the proviso that only one of X¹ or X² is C═O,
X³ is CHR′, NR′, O or S; 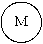
is a five- or six-membered heteroaromatic or six-membered aromatic ring optionally substituted by R¹¹;
R* is 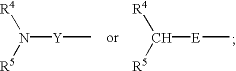
E is O, S, C(O) or C(S);
Y is C(O), C(S) or CH₂;
R¹ is R⁷, or D-C₀₋₄alkyl, D-C₂₋₄alkenyl, D-C₂₋₄alkynyl, D-C₃₋₄oxoalkenyl, D-C₃₋₄oxoalkynyl, D-C₁₋₄aminoalkyl, D-C₃₋₄aminoalkenyl, D-C₃₋₄aminoalkynyl, optionally substituted by any accessible combination of one or more of R¹¹ or R⁷;
R² is —(CH₂)ₜ—CO₂R³;
R³ is H, C₁₋₆alkyl or (CHR′)ₙ—Ar;
R⁴ is 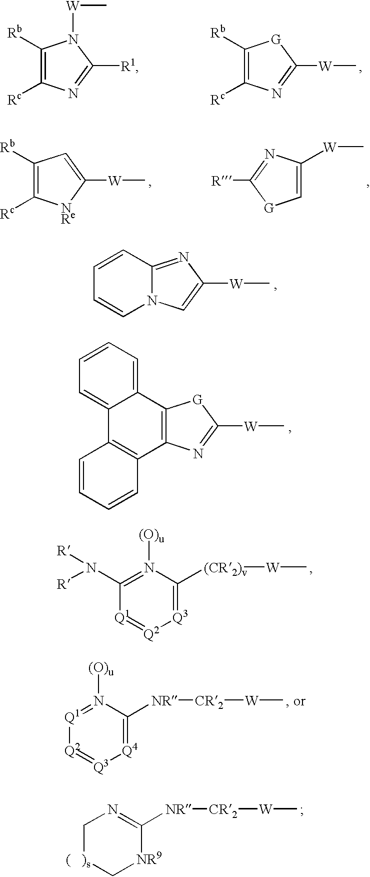
W is —(CHR^{g})ₐ-U-(CHR^{g})_{b}-;
U is absent or CO, CR^{g}₂, C(═CR^{g}₂), S(O)ₖ, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}, NR^{g}C(S), S(O)₂NR^{g}, NR^{g}S(O)₂ N═N, NR^{g}NR^{g}, NR^{g}CR^{g}₂, NR^{g}CR^{g}₂, CR^{g}₂O, OCR^{g}₂, C≡C or CR^{g}═CR^{g};
G is NR^{e}, S or O;
R^{g} is H, C₁₋₆alkyl, Het-C₀₋₆alkyl, C₃₋₇cycloalkyl-C₀₋₆alkyl or Ar—C₀₋₆alkyl;
R^{k} is R^{g}, —C(O)R^{g}, or —C(O)OR^{f};
Rⁱ is is H, C₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂;
R^{f} is H, C₁₋₆alkyl or Ar—C₁₋₆alkyl;
R^{e} is H, C₁₋₆alkyl, Ar—C₁₋₆alkyl, Het-C₁₋₆alkyl, C₃₀₇cycloalkyl-C₁₋₆alkyl, or (CH₂)ₖCO₂R^{g};
R^{b} and R^{c} are independently selected from H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂, or R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy;
Q¹, Q², Q³ and Q⁴ are independently N or C—R^{y}, provided that no more than one of Q¹Q², Q³ and Q⁴ is N;
R^{y} is H, halo, —OR^{g}, —SR^{g}, —CN, —NR^{g}R^{k}, —NO₂, —CF₃, CF₃S(O)ᵣ-, —CO₂R^{g}, —COR^{g} or —CONR^{g}₂, or C₁₋₆alkyl optionally substituted by halo, —OR^{g}, —SR^{g}, —CN, —NR^{g}R″, —NO₂, —CF₃, R′S(O)ᵣ-, —CO₂R^{g}, —COR^{g} or —CONR^{g}₂;
R⁵ is W′-(CR′₂)_{q}-Z-(CR′R¹⁰)ᵣ-U′-(CR′₂)ₛ;
U′ is absent or CO, CR′₂, C(═CR′2), S(O)ₙ, O, NR′, CR′OR′, CR′(OR″)CR′₂, CR′₂CR′(OR″), C(O)CR′₂, CR′₂C(O), CONR′, NR′CO, OC(O), C(O)O, C(S)O, OC(S), C(S)NR′, NR′C(S), S(O)ₙNR′, NR′S(O)ₙ, N═N, NR′NR′, NR′CR′₂, NR′CR′₂, CR′₂O, OCR′₂, c≡c or CR′═CR′;
W′ is R′R″N—, R′R″NR′N—, R′R″NR′NCO—, R′₂NR′NC(═NR′)—,
R′ONR′C(═NR′)—, 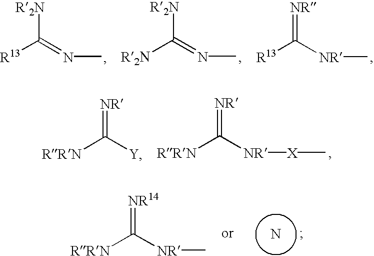
X is N═CR′, C(O) or O;
Y is absent, S or O;
Z is (CH₂)ₜ, Het, Ar or C₃₋₇cycloalkyl;
R⁷ is —COR⁸, —COCR′₂R⁹, —C(S)R⁸, —S(O)ₘOR′, —S(O)ₘNR′R″, —PO(OR′), —PO(OR′)₂, —B(OR′)₂, —NO₂ and Tet;
R⁸ is —OR′, —NR′R″, —NR′SO₂R′, —NR′OR′, —OCR′₂C(O)OR′, —OCR′₂OC(O)—R′, —OCR′₂C(O)NR′₂ or CF₃;
R⁹ is —OR′, —CN, —S(O)ᵣR′, S(O)ₘNR′₂, —C(O)R′C(O)NR′₂ or —CO₂R′;
R¹⁰ is H, C₁₋₄alkyl or —NR′R″;
each R¹¹ independently is H, halo, —OR¹², —CN, —NR′R¹², —NO₂, —CF₃, CF₃S(O)ᵣ-, —CO₂R′, —CONR′₂, D-C₀₋₆alkyl-, D-C₁₋₆oxoalkyl- , D-C₂₋₆alkenyl- , D-C₂₋₆alkynyl-, D-C₀₋₆alkyloxy-, D-C₀₋₆alkylamino- or D-C₀₋₆alkyl-S(O)ᵣ-;
R¹² is R′, —C(O)R′, —C(O)NR′₂, —C(O)OR¹⁵, —S(O)ₘR′ or S(O)ₘNR′₂;
R¹³ is R′, —CF₃, —SR′, or —OR′;
R¹⁴ is R′, C(O)R′, CN, NO₂, SO₂R′ or C(O)OR¹⁵;
R¹⁵ is H, C₁₋₆alkyl or Ar—C₀₋₄alkyl;
D is H, C₃₋₆cycloalkyl, Het or Ar;
R′ is H, C₁₋₆alkyl, Ar—C₀₋₆alkyl or C₃₋₆cycloalkyl-C₀₋₆alkyl;
R″ is R′, —C(O)R′ or —C(O)OR′;
R′″ is H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂;
n is 0 to 3;
q is 0 to 3;
t is 0 to 2;
a is 0, 1 or 2;
b is 0, 1 or 2;
k is 0, I or 2;
m is 1 or 2;
r is 0, 1 or 2;
s is 0, or 2;
u is 0 or 1; and
v is 0 or 1;
or a pharmaceutically acceptable salt thereof.

2. A compound according to formula (1a): 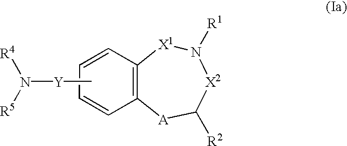
wherein:
A is NR′, CHR′, O or S;
X¹ and X² are C═O or CHR′, with the proviso that only one of X¹ or X² is C═O;
Y is C(O), C(S) or CH₂;
R¹ is R⁷, or D-C₀₋₄alkyl, D-C₂₋₄alkenyl, D-C₂₋₄alkynyl, D-C₃₋₄oxoalkenyl, D-C₃₋₄oxoalkynyl, D-C₁₋₄aminoalkyl, D-C₃₋₄aminoalkenyl, D-C₃₋₄aminoalkynyl, optionally substituted by any accessible combination of one or more of R¹¹ or R⁷;
R² is —(CH₂)ₜ—CO₂R³;
R³ is H, C₁₋₆alkyl or (CHR′)ₙ—Ar;
R⁴ is 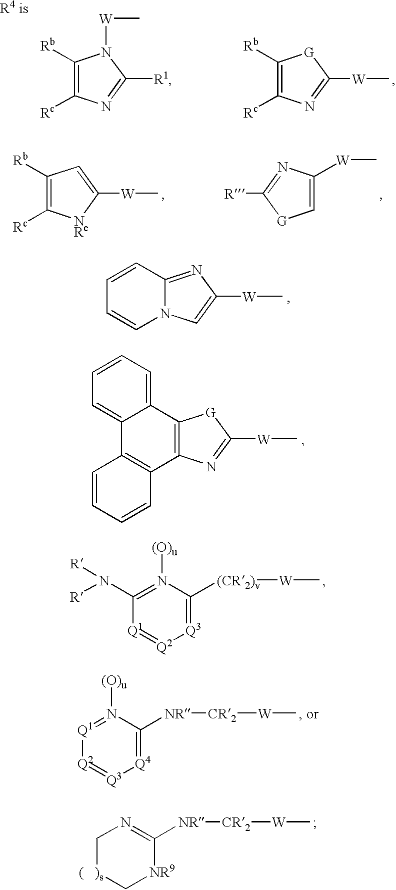
W is —(CHR^{g})ₐ—U— (CHR^{g})_{b}—;
U is absent or CO, CR^{g}₂, C(═CR^{g}₂), S(O)ₖ, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱ CO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, NR^{g}CR^{g}₂, CR^{g}₂O, OCR^{g}₂, C≡C or CR^{g}═CR^{g};
G is NR^{e}, S or O;
R^{g} is H, C₁₋₆alkyl, Het-C₀₋₆alkyl, C₃₋₇cycloalkyl-C₀₋₆alkyl or Ar—C₀₋₆alkyl;
R^{k} is R^{g}, —C(O)R^{g}, or —C(O)OR^{f};
Rⁱ is is H, C ₁₋₆alkyl, Het-C₀₋₆alkyl, C₃₋₇cycloalkyl-C₀₋₆alkyl, Ar—C₀₋₆alkyl, or C₁₋₆alkyl substituted by one to three groups chosen from halogen, CN, NR^{g}₂, OR^{g}, SR^{g}, CO₂R^{g}, and CON(R^{g})₂;
R^{f} is H, C₁₋₆alkyl or Ar—C₁₋₆alkyl;
R^{e} is H, C₁₋₆alkyl, Ar—C₁₋₆alkyl, Het-C₁₋₆alkyl, C₃₋₇cycloalkyl-C₁₋₆alkyl, or (CH₂)^{k}CO₂R^{g};
R^{b} and R^{c} are independently selected from H, C₁₋₆alkyl. Ar—C₀₋₆alkyl, Het-C₀₋₆alkyl, or C₃₋₆cycioalkyl-C₀₋₆alkyl, halogen, CF₃, OR^{f, S(O)}ₖR^{f}, COR^{f}, NO₂, N(R^{f})₂, CO(NR^{f})₂, CH₂N(R^{f})₂, or R^{b} and R^{c} are joined together to form a five or six membered aromatic or non-aromatic carbocyclic or heterocyclic ring, optionally substituted by up to three substituents chosen from halogen, CF₃, C₁₋₄alkyl, OR^{f}, S(O)ₖR^{f}, COR^{f}, CO₂R^{f}, OH, NO₂, N(R^{f})₂, CO(NR^{f})₂, and CH₂N(R^{f})₂; or methylenedioxy;
Q¹, Q², Q³ and Q⁴ are independently N or C—R^{y}, provided that no more than one of Q¹, Q², Q³ and Q⁴ is N;
R^{y} is H, halo, —OR^{g}, —SR^{g}, —CN, —NR^{g}R^{k}, —NO₂, —CF₃, CF₃S(O)ᵣ-, —CO₂R^{g}, —COR^{g} or —CONR^{g}₂, or C₁₋₆alkyl optionally substituted by halo, —OR^{g}, —SR^{g}, —CN, —NR^{g}R″, —NO₂, —CF₃, R′S(O)^{r}—, —CO₂R^{g}, —COR^{g} or —CONR^{g}₂;
R⁵ is W′-(CR′₂)_{q}-Z-(CR′R¹⁰)ᵣ-U′-(CR′₂)ₛ;
U′ is absent or CO, CR′₂, C(═CR′2), S(O)ₙ, O, NR′, CR′OR′, CR′(OR″)CR′₂, CR′₂CR′(OR″), C(O)CR′₂, CR′₂C(O), CONR′, NR′CO, OC(O), C(O)O, C(S)O, OC(S), C(S)NR′, NRC(S), S(O)ₙNR′, NR′S(O)ₙ, N═N, NR′NR′, NR′CR′₂, NR′CR′₂, CR′₂O, OCR′₂, C≡C or CR′═CR′;
W′ is R′R″N—, R′R″NRN—, R′R″NR′NCO—, R′₂NR′NC(═NR′)—,
R′ONR′C(═NR′)—, 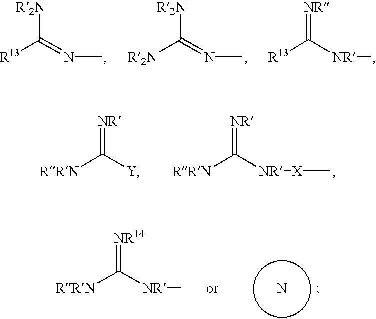
X is N═CR′, C(O) or O;
Y is absent, S or O;
Z is (CH₂)ₜ, Het, Ar or C₃₋₇cycloalkyl;
R⁷ is —COR⁸, —COCR′₂R⁹, —C(S)R⁸, —S(O)ₘOR′, —S(O)ₘNR′R″, —PO(OR′), —PO(OR′)₂, —B(OR)₂, —NO₂ and Tet;
R⁸ is —OR′, —NR′R″, —NR′SO₂R′, —NR′OR′, —OCR′₂C(O)OR′, —OCR′₂OC(O)— R′, —OCR′₂C(O)NR′₂ or CF₃;
R⁹ is —OR′, —CN, —S(O)ᵣR′, S(O)ₘNR′₂, —C(O)R′C(O)NR′₂ or —CO₂R′;
R¹⁰ is H, C₁₋₄alkyl or —NR′R″;
R¹¹ is H, halo, —OR¹², —CN, —NR′R¹², —NO₂, —CF₃, CF₃S(O)ᵣ, —CO₂R′, —CONR′₂, D-C₀₋₆alkyl-, D-C₁₋₆oxoalkyl-, D-C₂₋₆alkenyl-, D-C2-6alkynyl-, D-C₀₋₆alkyloxy-, D-C₀₋₆alkylamino- or D-C₀₋₆alkyl-S(O)ᵣ-;
R¹² is R′, —C(O)R′, —C(O)NR′₂, —C(O)OR¹⁵, —S(O)ₘR′ or S(O)ₘNR′₂;
R¹³ is R′, —CF₃, —SR′, or —OR′;
R¹⁴ is R′, C(O)R′, CN, NO₂, SO₂R′ or C(O)OR¹⁵;
R¹⁵ is H, C₁₋₆alkyl or Ar—C₀₋₄alkyl;
D is H, C₃₋₆cycloalkyl, Het or Ar;
R′ is H, C₁₋₆alkyl, Ar—C₀₋₆alkyl or C₃₋₆cycloalkyl-C₀₋₆alkyl;
R″ is R′, —C(O)R′ or —C(O)OR′;
R′″ is H, C₁₋₆alkyl, Ar—C₀₋₆alkyl, Het-C₀₋₆alkyl, or C₃₋₆cycloalkyl-C₀₋₆alkyl, halogen, CF₃, OR^{f}, S(O)ₖR^{f}, COR^{f}, NO₂, N(R^{f})₂, CO(NR^{f})₂, CH₂N(R^{f})₂;
n is 0 to 3;
q is 0 to 3;
t is 0 to 2;
a is 0, 1 or 2;
bis 0, 1 or2;
k is 0, 1 or 2;
m is 1 or 2;
r is 0, 1 or 2;
s is 0, 1 or 2;
u is 0 or 1; and
v is 0 or 1;
or a pharmaceutically acceptable salt thereof.

3. The compound according to claim 2 wherein Y is C(O).

4. The compound according to claim 2 wherein X¹ is CH₂ and X² is C═O.

5. The compound according to claim 2 wherein A is NR′, in which R′ is H or C₁₋₄alkyl.

6. The compound according to claim 5 wherein R¹ is D-C₀₋₄alkyl, optionally substituted by R¹¹.

7. The compound according to claim 6 wherein R¹ is H, CH₃, CH₂CH₃, CH₂CF₃, (CH₂)₁₋₂phenyl, in which the phenyl group is unsubstituted or substituted by C₁₋₄alkyl, C₁₋₄alkoxy, C₁₋₄alkthio, CF₃, OH, or halo.

8. A compound according to claim 2 wherein R⁵ is chosen from: 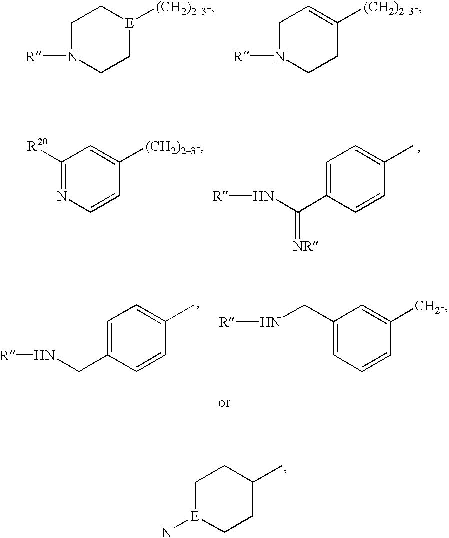
R″HNC(═NH)NH—(CH₂)₃(CHR¹⁰)—, and R″HN—(CH₂)₅—, wherein E is N or CH, and R²⁰ is hydrogen, amino, mono or di-C₁₋₄alkylamino, hydroxy or C₁₋₄alkyl.

9. A compound according to claim 8 wherein R⁵ is 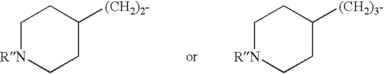
in which R″ is H.

10. A compound according to claim 2 wherein the group attached to W in R⁴ is chosen from: 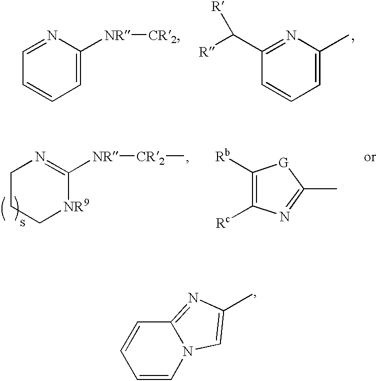
wherein G is NH and R^{b} and R^{c} are joined to form a cyclohexyl, phenyl or pyridyl ring and R′, R″ and R^{g} are each H and s is 0 or 1.

11. A compound according to claim 2 wherein R⁴ is:

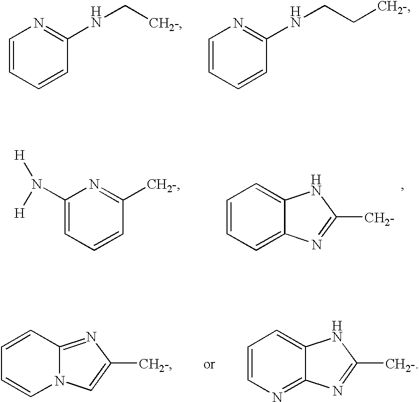

12. A compound according to claim 11 wherein R⁴ is:

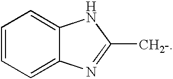

13. A compound according to claim 2 which is:
(S)-7-[[N-(4-pyridinylpropyl)-N-(1H-benzimidazol-2-ylmethyl)]-aminocarbonyl]-2,3,4,5-tetrahydro-4-methyl-3-oxo-1H-1,4-benzodiazepine-2-acetic acid;
(S)-7-[[N-4-piperidinylpropyl-N-(1H-benzimidazol-2-ylmethyl)]-aminocarbonyl]-2,3,4,5-tetrahydro-4-methyl-3-oxo-1H-1,4-benzodiazepine-2-acetic acid;
(S)-7-[[N-(4-piperldinylpropyl)-N-(1H-4,5,6,7-tetrahydro-benzimidazol-2-ylmethyl)]aminocarbonyl ]-2,3,4,5-tetrahydro-4-methyl-3-oxo-1H-1,4-benzodiazepine-2-acetic acid; or
(+/−)-2,3,4,5-tetrahydro-3-oxo-8-[[N-4-piperidinyfpropyl-N-(1H-benzimidazol-2-ylmethy]aminocarbonyl]-2-methyl-1H-2-benzazepine-4-acetic acid;
or a pharmaceutically acceptable salt thereof.

14. A pharmaceutical composition comprising a compound according to any one of claims 1-13 and a pharmaceutically acceptable carrier.

15. A method of inhibiting the fibrinogen receptor and the vitronectin receptor comprising administering a compound according to any one of claims 1-13.

16. A method of for treating atherosclerosis or restenosis following angioplasty in a mamnmal comprising admiinistering, a compound according to any one of claims 1-13.

17. A compound according to any one of claims 1 to 13 for use as a medicament.

18. The use of a compound as defined in any one of claims 1 to 13 in the manufacture of a medicament for the treatment of diseases in which the inhibition of the fibrinogen receptor and the vitronectin receptor is indicated.

19. The use of a compound as defined in any one of claims 1 to 13 in the manufacture of a medicament for the treatment of atherosclerosis or restenosis.
